(19) United States
(12) Patent Application Publication
(10) Pub. No.: US 2021/0177085 A1
(43) Pub. Date: Jun. 17, 2021

(54) SHOE WITH TRACKING DEVICE AND MEDICATION ALERT

(71) Applicant: SHERRIE ANN SANDERS, RINCON, GA (US)

(72) Inventor: SHERRIE ANN SANDERS, RINCON, GA (US)

(21) Appl. No.: 17/188,796

(22) Filed: Mar. 1, 2021

Related U.S. Application Data
(63) Continuation-in-part of application No. 14/535,602, filed on Nov. 7, 2014.

(51) Int. Cl. A43B 3/00; A43B 1/00; H04W 4/029; H04W 4/02

(52) U.S. Cl. CPC A43B 3/0005; H04W 4/023; H04W 4/029; A43B 1/0054

(57) ABSTRACT

The present invention discloses a shoe with a tracking device. The tracking device may allow for tracking the shoe. The shoe may include more than one tracking device on the shoe. The show may alert a user of the shoe with a vibration and/or words “med time” when it is time for the user to take medication.

### BACKGROUND OF THE INVENTION

The present invention relates to the field of footwear, and more particularly to the field of human footwear. This application claims the benefit of application Ser. No. 14/535,602 filed Nov. 7, 2014.

### SUMMARY OF THE INVENTION

In one embodiment the invention, a tracking shoe device, comprises a shoe; and a first tracking device attached to an interior front portion of the shoe.

In a further embodiment of the invention, a method comprises attaching a first tracking device to an interior front portion of the shoe; and tracking the shoe using the first tracking device to track the shoe.

### DESCRIPTION OF THE FIGURES

FIG. 1 is a side view of an embodiment of the invention.

FIG. 2 is a side view of an another embodiment of the invention.

FIG. 3 is a bottom view of the embodiment from FIG. 1.

FIG. 4 is a bottom view of the embodiment from FIG. 2.

FIG. 5 is a side view of the invention with two tracking devices.

FIG. 6 is a side view of another embodiment of the invention of FIG. 5 with the first tracking device attached to a tongue of the shoe.

FIG. 7 is a side view of another embodiment of the invention of

FIG. 5, with the first tracking device attached to an interior front of the shoe.

FIG. 8 is a side view of another embodiment of the invention of FIG. 5, with the tracking devices attached with magnets.

FIG. 9 is a side view of another embodiment of the invention of

FIG. 5, with the tracking devices camouflaged.

### DETAILED DESCRIPTION

The following description is not intended to limit the scope of the invention, but to generally illustrate the invention.

A shoe with a tracking device can aid in tracking persons, such as persons with dementia.

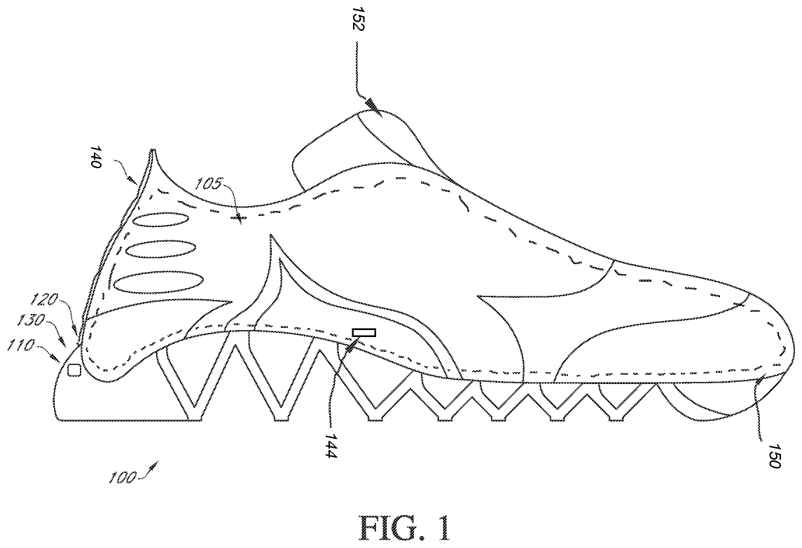

FIG. 1 is a front view of the present invention 100. The invention 100 may include a shoe 105 with a first tracking device 110. In an embodiment, the first tracking device 110 may be a global positioning system (GPS) tracking device. In an embodiment, the first tracking device 110 may be located on an upper back 130 of a heel 120 of the shoe 105. In an example, the tracking device 110 may alert authorities and family members when a wearer of the shoe 105 has wandered off a significant distance from a primary residence or designated location. Once the alert notification has been activated, authorities may organize a search and recovery team. The team may take any and all precautionary measures to ensure that the individual is safely returned while working closely with local law enforcement and first responders. After the individual is safely returned, the alert notification may be reset and reprogrammed. This may be accomplished by integrating the tracking device 110 with the shoe 105.

In an embodiment, the invention 100 may use Velcro 152 to secure the shoe 105. In an embodiment, the invention 100 may have a purple strip 140 at the back of the shoe 105, which helps to alert persons or vehicles as to proximity to the shoe 105. In an embodiment, the invention 100 may have memory foam 150 along the entire inside of the shoe, both top and bottom, which helps add comfort to a user of the shoe 105. In an embodiment, the invention 100 may include a device 144 that vibrates at a time when a user needs to take medication. The device 144 may also make the sound “med time” at the time the user needs to take medication in addition to vibrating. The user of the shoe 105 may feel the vibrating and may also hear the sound “med time” at a settable time. The device 144 may be programmable for a particular time of day to vibrate and/or emit the “med time” sound.

Moreover, the invention 100 may be efficient for consumers within certain miles radius and different elements such as water, wood area, and snow. The invention 100 may be used by group and nursing homes, assisted living facilities, retirement communities, and hospitals. This may afford the family members the opportunity to locate their loved ones at a moment notice, if they wondered off from their designated location by ensuring their safety.

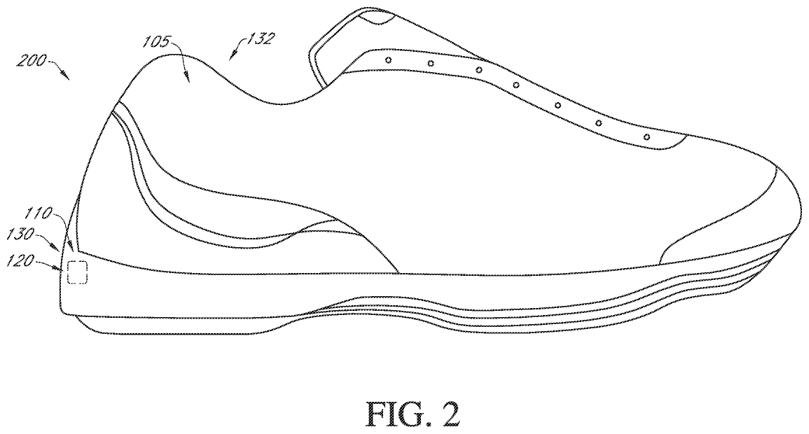

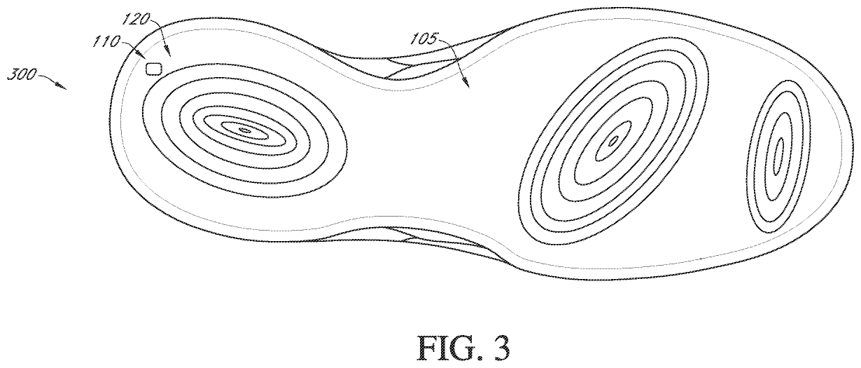

FIG. 2 is a side view of another embodiment 200 of the present invention. A first tracking device 110 is shown placed on an interior 132 of an upper back 130 of the heel 120 of the shoe 105.

FIG. 3 is a bottom view 300 of the embodiment from FIG. 1. Shown is the first tracking device 110 in the heel 120 of the shoe 105.

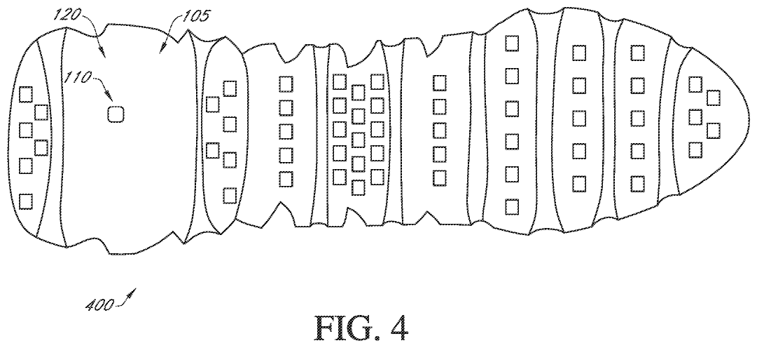

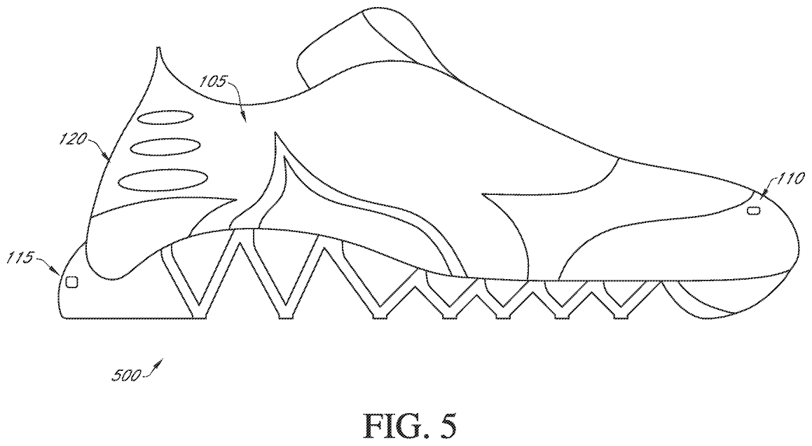

FIG. 4 is a bottom view 400 of the embodiment from FIG. 2. Shown is the first tracking device 110 in the heel 120 of the shoe 105.

FIG. 5 shows an embodiment 500 of the present invention 100 with a first tracking device 110, and a second tracking device 115. The first and second tracking devices may operate individually or simultaneously.

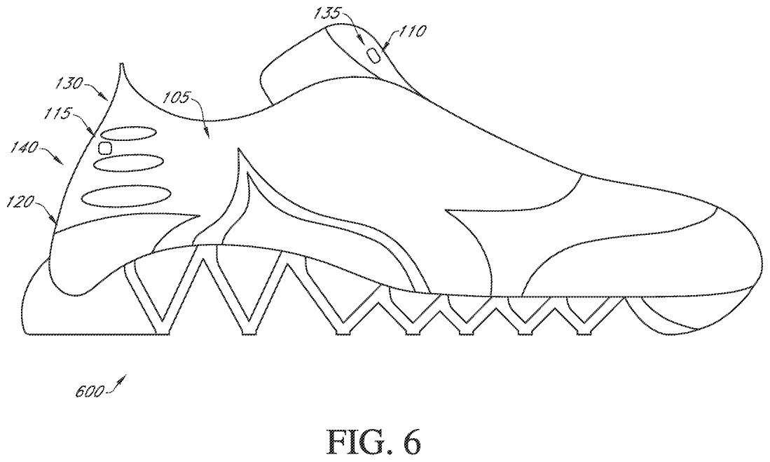

FIG. 6 shows an embodiment 600 of the present invention with the first tracking device 110 attached to a tongue 135 of the shoe 105 and the second tracking device 115 on an exterior 140 of the upper back 130 of the heel 120 of the shoe 105.

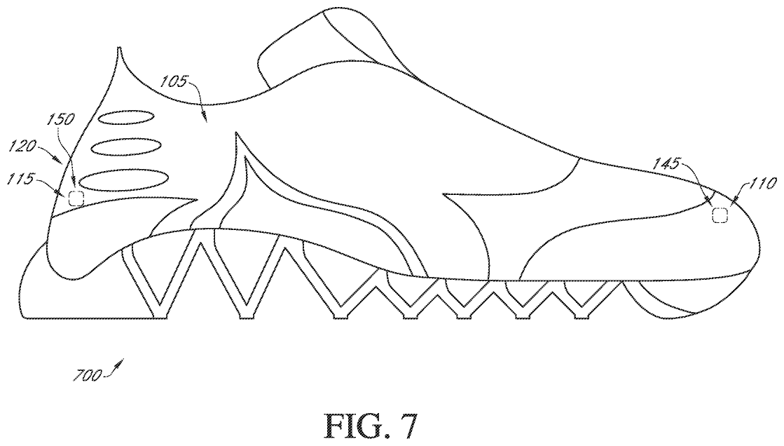

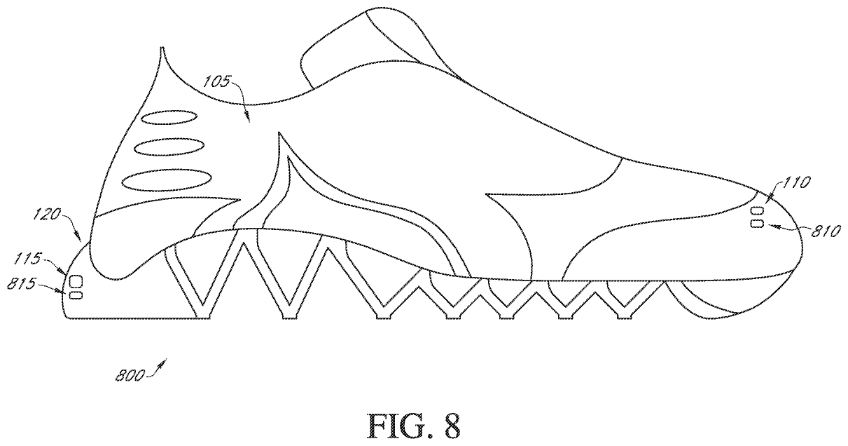

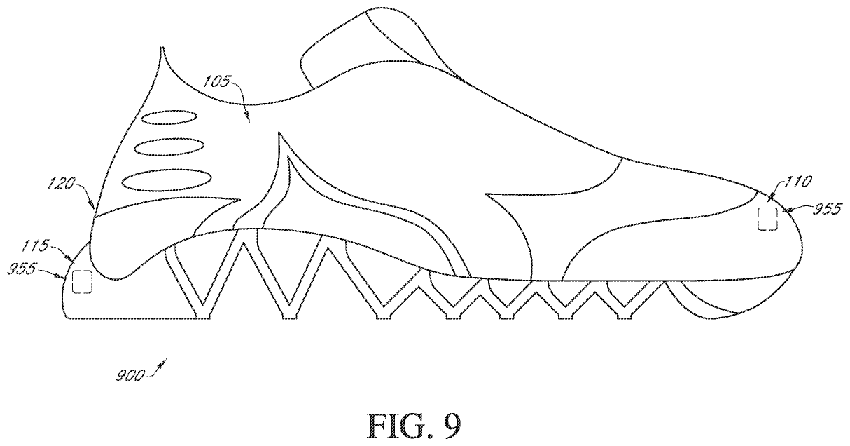

FIG. 7 shows an embodiment 700 of the present invention with the first tracking device 110 attached to an interior front 145 of the shoe 105. The second tracking device 115 is shown attached to the interior back 150 of the heel 120 of the shoe 105.

FIG. 8 shows an embodiment 800 of the invention of FIG. 1 with the first tracking device 110 and the second tracking device 115 attached with magnets 810, 815, with a magnet 810 securing the first tracking device 110 to the shoe 105, and a second magnet 815 securing the second tracking device 115 to the shoe 105.

FIG. 9 shows an embodiment 900 of the invention of FIG. 1 with the first tracking device 110 camouflaged and disguised with shoe material 955 as part of the shoe 105. The second tracking device 115 is also camouflaged and disguised with shoe material 955 as part of the shoe 105.

The invention may be made of any suitable material including but not limited to, cloth products, plastic products, rubber products, and other natural or human made materials. The invention may be created with only the first tracking device, only the second tracking device, or with both tracking devices. The first tracking device 110 and the second tracking device 115 may also be attached to a side edge of the shoe 105.

The above description may be modified without departing from the scope of the claims below.

We claim:

1. A tracking shoe device, comprising:
a shoe; and
a first tracking device attached to a first part of the shoe, wherein the first tracking device is a global positioning system (GPS) tracking device,
wherein the first tracking device is camouflaged with shoe material to look like the first part of the shoe,
a second tracking device connected to the shoe on another end of the shoe from the first tracking device,
wherein the second tracking device is camouflaged with shoe material to look like a second part of the shoe,
wherein the first tracking device and the second tracking device are attached with a magnet to the shoe; and
wherein the first tracking device and the second tracking device alert both authorities and family members with a programmable alert signal when a wearer of the shoe is a distance from a designated location,
wherein the programmable alert signal is capable of being reset after the first tracking device and the second tracking device are successfully tracked by the authorities;
wherein the shoe includes utilizes Velcro® to attach the shoe;
wherein the shoe includes a purple strip attached to a back of the shoe;
wherein the shoe includes memory foam along the entire inside of the shoe, both top and bottom; and
wherein the shoe includes a device that vibrates at a programmable time and emits the sound “med time” at the programmable time.
